(19) United States
(12) Patent Application Publication
(10) Pub. No.: US 2024/0417735 A1
(43) Pub. Date: Dec. 19, 2024

(54) ORAL CODELIVERY OF 5-FLUOREURACIL AND Bcl2 siRNA TREATING STOMACH CANCER

(71) Applicant: Board of Regents, The University of Texas System, Austin, TX (US)

(72) Inventors: Md Nurunnabi, El Paso, TX (US); Humayra Afrin, El Paso, TX (US); Raj Kumar, El Paso, TX (US)

(21) Appl. No.: 18/742,639

(22) Filed: Jun. 13, 2024

Related U.S. Application Data
(60) Provisional application No. 63/507,935, filed on Jun. 13, 2023.

(51) Int. Cl. C12N 15/113; A61K 9/00; A61K 9/51; A61K 31/513; A61P 35/00

(52) U.S. Cl. CPC C12N 15/1135; A61K 9/006; A61K 9/5123; A61K 31/513; A61P 35/00; C12N 2310/14

(57) ABSTRACT

Compositions and methods are described for oral treatment of stomach cancer. A composition of matter for treatment of stomach cancer includes fluoreuracil-5; and Bcl2 siRNA. The composition can include β-glucan as a mucoadhesive nanocarrier.

### CROSS REFERENCE TO RELATED APPLICATION

Referring to the application data sheet filed herewith, this application claims a benefit of priority under 35 U.S.C. 119(e) from co-pending provisional patent application U.S. Ser. No. 63/507,935, filed Jun. 13, 2023, the contents of which are incorporated herein by reference in their entirety for all purposes.

### STATEMENT AS TO RIGHTS TO INVENTIONS MADE UNDER FEDERALLY-SPONSORED RESEARCH OR DEVELOPMENT

This invention was made with government support under Award No R03D032624 awarded by National Institutes of Health (NIH). The government has certain rights in the invention.

### BACKGROUND INFORMATION

### 1. Field

The present invention relates generally to the field of medicine and disease treatment. More particularly, it concerns methods and compositions for oral therapy of stomach cancer.

### 2. Background

Stomach cancer is the 5^{th} most common cancer by incidence and 3^{rd} most deadly cancer. In 2018, 782,685 out of 1,033,701 patients were died of stomach cancer worldwide. According to the data from the American Cancer Society 26,380 new stomach cancer patients will be diagnosed solely in the United States in 2022, and among the diagnosed individual, 11,090 will die. Currently available therapeutic interventions for stomach cancer treatment are chemotherapy, immunotherapy, radiotherapy, and surgery. Among FDA approved chemotherapeutic drugs, 5-fluoreuracil (5FU), Capecitabine, Docetaxel, Epirubicin, Doxorubicine, Trastuzumab, Trifluridine, and Tipiracil are most widely considered in clinic for stomach cancer treatment. Other promising drugs are those developed for immunotherapy, such as PDL1 inhibitors like Pembrolizumab or Nivolumab, and targeted gene therapy drugs have also shown promise for stomach cancer therapy. Some genes (for instance IL-8, CLDN1, KRT17, CLDN7, and MMP7) are reportedly upregulated, and some other genes (for instance GAS5, ZIC1, RASAL1, GPER, KIAA1324, ADA and SLC9A2) are found to be downregulated in stomach cancer. It has also been reported that upregulation of STAB1, STAT3, Bcl-2, and Bcl-xL genes occur in the stomach during cancerous state.

Heretofore, the requirements of attenuating stomach cancer while avoiding side effects and other undesirable consequences have not been fully met. In view of the foregoing, there is a need in the art for a solution that simultaneously solves all of these problems.

### SUMMARY

Based on cancer related deaths, stomach cancer is ranked 5^{th}, and 1^{st} among Hispanics. Lack of technologies for early diagnosis and unavailability of target specific therapeutics is largely the cause of the poor therapeutic outcomes from existing chemotherapeutics. Currently available therapeutic modalities are invasive and require systemic delivery, though the cancer is localized in the stomach at its early stage. Therefore, an oral local delivery approach can extend retention duration of the therapeutics modalities within the stomach and thereby enhance therapeutic efficacy. To accomplish this, we have developed a β-glucan (BG) based oral delivery vehicle that can adhere to the mucus lining of the stomach for an extended period while controlling release of Bcl2 siRNA and 5-fluoreuracil (5FU) payload for over 6 hr. We found that Bcl2 siRNA selectively knocked down the Bcl2 gene in C57BL/6 stomach cancer mice model followed by upregulation of apoptosis and remission of cancer. BG was found to be very effective in maintaining stability of siRNA for at least 6 hours, when submerged in simulated gastric juice tested in vitro. To investigate potential therapeutic effects in vivo, we used a stomach cancer mice model, where C57BL/6 mice were treated with 5FU, BG/5FU, siRNA, BG/siRNA, and BG/5FU/siRNA. Higher inhibition of Bcl2 and therapeutic efficacy was observed in mice treated with BG/5FU/siRNA confirmed with Western blotting and a tunnel assay. Significant reduction in tumor region was observed with histology (H&E) and immunohistochemistry (Ki67, TUNEL, and Blc2) analysis. In conclusion, we have developed an oral formulation for delivery of stomach cancer therapeutics that shows improved efficacy with non-significant side effects compared to the conventional treatment.

Stomach specific oral delivery of anticancer therapeutics can enhance the retention duration of the therapeutic modalities thus improving the therapeutic effect by increasing drug concentration within the site of cancer. 5FU is the first-line treatment of choice for stomach cancer, however, toxic effect and development of resistance are the major concern. Considering the risk to benefit ratio of using 5FU, there is a concern about their broad-spectrum application for various cancer. In this study, oral delivery of 5FU and Bcl2 siRNA were used to enhance therapeutic effect by synergistic effect of 5FU and siRNA. RNAi is gaining popularity as an anti-cancer treatment option because of selective gene silencing and no off-targeting effect which provides better therapeutic efficacy and fewer side effect. To overcome the challenges associated with the oral delivery of siRNA, we have developed a nano carrier composed of BG, a highly biocompatible polysaccharide. The BG was not only protecting the payload of siRNA to maintain their stability but enhancing their retention within the stomach for at least 6 hr. The co-delivery of BG/5FU/siRNA was shown to downregulate Bcl2 expression and apoptosis of cancer cells. Ki67 marker was also reduced with the treatment and histological staining shows almost complete remission of the tumor region with BG/5FU/siRNA formulation. This study provides an oral therapeutic modality for stomach cancer treatment which is more effective than free siRNA and only 5FU. However, the pathway of producing a synergistic effect in silencing Bcl2 has not been examined in this study. Therefore, we aim to continue this study to understand the mechanism of action of 5FU and Bcl2 siRNA combination. As this study provided a platform for successful oral siRNA delivery which creates a new horizontal for oral delivery of other biological therapeutics.

An illustrative embodiment of the present disclosure is a composition of matter for oral treatment of stomach cancer, comprises fluoreuracil-5; and Bcl2 siRNA.

Another illustrative embodiment of the present disclosure is a method of treating stomach cancer comprising administering to a mammal in need thereof a compound comprising fluoreuracil-5; and Bcl2 siRNA.

Another illustrative embodiment of the present disclosure is a method of orally treating disease comprising administering to a mammal in need thereof a compound comprising fluoreuracil-5; and Bcl2 siRNA.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates the mechanism of action of co-treatment of 5-FU and Bcl2 siRNA and their synergy to improve stomach cancer treatment in accordance with an illustrative embodiment.

FIGS. 2A-2E illustrate chemical and physical properties of the formulations in accordance with an illustrative embodiment. (A) Chemical structure (repeating unit) and schematic presentation of BG. (B) Schematic representation of preparation of formulations; BG/siRNA, BG/5FU and BG/5FU/siRNA. (C) SEM images of BG/5FU/siRNA BG showed smooth surface and nano size (Scale bar=5 μm). (D) Size distribution. (E) Zeta potential of all the formulations were measured using DLS and zeta analyzer.

FIGS. 3A-3D illustrate in vivo therapeutic efficacy in accordance with an illustrative embodiment. (A) Schematic of animal model preparation and treatment. B) Image of the stomach collected from different group of treatment—Healthy (a), Untreated cancer (c), BG/5FU (c), 5FU (d), siRNA (e), BG/siRNA (f), BG/5FU/siRNA (g). (C) Weight of the stomach. (D) Size of the stomach.

FIGS. 4A-4D illustrate immunohistochemistry to attribute apoptosis in accordance with an illustrative embodiment. (A) Tunnel assay shows broken nucleus (arrow) in siRNA, BG/siRNA, and BG/5FU/siRNA groups. Scale bars represent 200 μm. (B) Western blot analysis also shows reduced Bcl-2 expression in BG/5FU/siRNA group compared to naked siRNA and BG/siRNA. The error bar represents the standard deviation. (C) Quantitative analysis of WB. (D) Apoptosis in percentage. P value<0.0001 is denoted as ***.

FIGS. 5A-5B illustrate immunohistochemistry to attribute expression of Bcl2 protein in accordance with an illustrative embodiment. (A) Immunohistochemistry for Bcl-2 protein shows higher expression of Bcl-2 in untreated mice stomachs (arrow). The naked oral siRNA treatment group also had more Bcl-2 expression than BG/siRNA and BG/5FU/siRNA. The scale bar represents 100 μm. (B) Quantification of Bcl2 immunofluorescence data analysis.

FIGS. 6A-6B illustrate immunofluorescence for ki67 of stomach sections of different treatment groups in accordance with an illustrative embodiment. (A) A higher expression of ki67 (yellow arrow) was observed in untreated group, whereas after treatment the expression got reduced specially with BG/5FU/siRNA formulation. Scale bar represents 100 μm. (B) Ki67 expression was quantified compared to healthy control.

FIGS. 7A-7B illustrate histology of stomach. (A) The histological staining (H&E) shows a growth (arrow) in the untreated section, while after treatment with 5FU, 5FU/BG and BG/5FU/siRNA the tumor region got significantly reduced. (B) H&E staining of stomach cancer sections with 40× magnification. The rectangular area was cropped and magnified for better morphological understanding. The untreated, naked siRNA and BG/siRNA shows the feature of adenocarcinoma. The section of BG/5FU/siRNA treatment group almost resemblance as healthy stomach in accordance with an illustrative embodiment.

### DETAILED DESCRIPTION

The disclosure of this application is technically related to co-pending U.S. Ser. No. ______ (attorney docket number UTEP2023-013-2), filed Jun. 13, 2024, the entire contents of which are hereby expressly incorporated by reference for all purposes.

Embodiments of this disclosure include a novel oral drug delivery vehicle that can facilitate oral delivery of anticancer drug, enhance retention duration within the stomach to maintain therapeutic dose concentration within the cancer area, and thereby enhance cancer treatment with minimal toxicities/side effect. Embodiments of this disclosure can solve problems related to stomach cancer treatment. Heretofore, there are few technologies for oral medications for stomach cancer treatment. Embodiments of this disclosure can provide benefits for individual who are scared of needles because embodiments of this disclosure can include a painless oral medication approach. Embodiments of this disclosure will be beneficial for every stomach cancer patient who would otherwise go through chemotherapy and experience tremendous toxicities.

Medications according to embodiments of this disclosure can be administered orally instead of injection. The medication can reach the cancer site within a very short period of time as administered orally, and more importantly the medication can maintain retention and concentration within stomach for at least 6 hours. Consequently, a lesser dose will provide higher therapeutic effect and thereby we will be able to minimize anticancer drug mediated toxicities for other organs including heart, lung, and liver. Embodiments of this disclosure can include co-delivery of 2 individual modalities, for instance 5 flurouracil in combination with siRNA. This combination delivery approach will provide synergistic therapeutic effect that is better than the single modalities.

Like the treatment of other cancers, for stomach cancer treatment, dosage form, rout of administration and dosage amount of the chemotherapeutics is crucial to avoid potential severe risk of toxicity to the healthy tissue associated with non-specific distribution. Stomach cancer management is severely challenging due to the difficulty of delivering the appropriate amount of drug to the location of cancer and maintain their therapeutic concentration for prolong duration, regardless of rout of administration. This is further compounded by the relatively late diagnosis that is more typical of stomach cancer patients and that occurs when the cancer has grown and spread across the stomach. In addition, oral administration of the chemotherapeutics is not very effective due the bowel movement and shorter retention duration. The retention duration of any orally administered drug to the stomach is 10-30 min, much less than the duration often needed to induce adequate therapeutic effect and maintain appropriate therapeutic concertation within the cancer site. Finally, oral delivery of therapeutic modalities is challenging because of significantly low bioavailability resulting from poor intestinal permeability, enzymatic degradation, thick mucus membrane barrier, and off-target localization. In order to overcome these issues, we aim to develop an oral local delivery vehicle, made from the mucoadhesive carrier, b-glucan (BG). We have previously reported that BG is very stable in acidic buffer, has a strong binding affinity with mucin, and forms microstructure particles in presence of fatty acid. Studies show that the BG-based vehicle is highly effective in oral vaccine and protein delivery. The BG-mediated mucoadhesive particle has the ability to protect the therapeutic regimens from the harsh gastric environment, enhance retention of the therapeutic payload within the stomach for a longer duration, and maintain sufficient therapeutic concentration for prolong period (6 hours or more). In addition, BG was found to act as an immune modulator of the gastrointestinal (GI) tract. Finally, BG also has shown significant potential in GI site-specific drug delivery to treat liver diseases, bowel disease, and stomach cancer.

5FU is a pyrimidine analog, a potent and widely used chemotherapy to treat GI-tract related cancers including stomach cancer. A meta-analysis of stomach cancer treatment revealed a significant advantage in terms of 3-year survival when on chemotherapy with S-1, an oral 5FU derivative (80%), compared to the surgery (70%). 5FU or S-1 is considered as first-line treatment due to better efficacy but is recommended to administer with adequate precaution to avoid the severe risk of side effects associated with toxicity. Despite having higher therapeutic effectivity, oral delivery causes various side effects within the GI tract and intravenous administration produces toxicities in major organs including the heart, lungs and kidney. Side effects of 5FU include cardiotoxicity, hypotension, breathing problem, hair loss, and weight loss. With the development of a delivery platform that has the potential to minimize systemic distribution of 5FU and increase drug concentration within the site of cancer, it is highly likely possible to minimize drug-mediated toxicity and increase therapeutic effects. Besides, 5FU itself is not very effective of treating cancer, for instance, treatment response rate is between 10-15% for advanced colorectal cancer. Therefore, we presume that a combination of RNAi can improve the therapeutic effect by synergizing the mechanism of action. Therefore, we aim to incorporate a Bcl2 small interfering RNA (siRNA) that holds emerging promise for the genetic treatment of cancer. Since its discovery, siRNA is considered to apply for various cancer treatments as gene therapy including stomach cancer.

Bcl2 overexpression has been found in cancers including the stomach, breast and pancreas, where it promotes the proliferation of cancer cell. Bcl2 downregulation has also been to induce apoptosis to tumor cell and make them more sensitive to chemotherapy. Some recent reports have demonstrated strategies for improved oral delivery of siRNA to ameliorate hepatic cancer through nanoparticle and mucoadhesive molecules. The Bcl2 family is composed of a subset of proteins that act as apoptosis regulators. Research suggested that the hyperactivation of Bcl2-related anti-apoptotic effects correlates with cancer occurrence, progression, and prognosis. Therefore, we hypothesize that a combination of Bcl2 siRNA and 5FU can accelerate stomach cancer therapeutic outcomes when given orally.

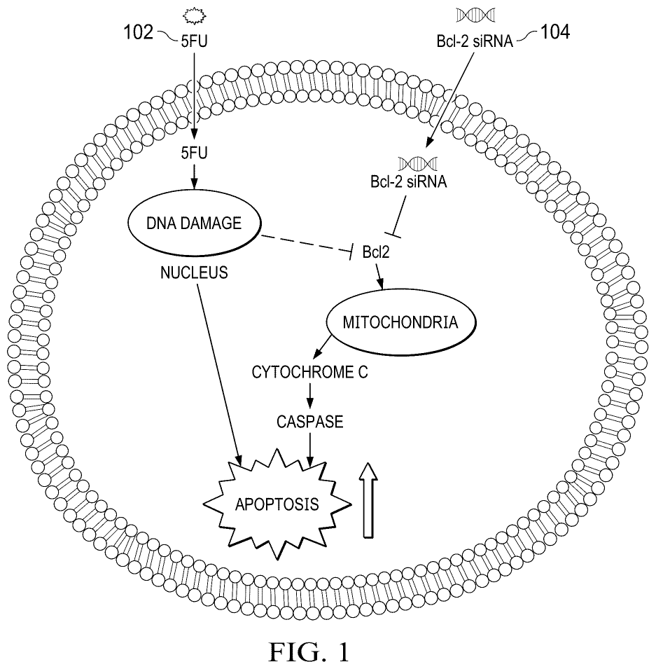

FIG. 1 shows the mechanism of action of co-treatment of 5-FU 102 and Bcl2 siRNA 104 and their synergy to improve stomach cancer treatment.

To prove the concept and to investigate therapeutic feasibility, 5FU and siRNA were loaded within a nanoparticle composed with BG. Both physical and chemical characterization were conducted prior to utilizing the formulation for in vitro and in vivo studies. We have observed that the BG-based oral vehicles not only increase retention of the payload of 5FU and siRNA within the stomach but also facilitate sustained release of the payload for over 6 hr. In vivo studies revealed that the oral delivery of BG/siRNA/5FU downregulates the Bcl2 protein by 60% and increases apoptosis by 140% with a daily oral dose for 15 days. Immunohistochemistry analysis of stomach tissue also shows that Ki67 expression reduces by 300% compared to untreated cancer and 5FU groups, and 200% compared to siRNA treated group. These results are obvious indication of the potency and effectiveness of the BG-based oral formulation of siRNA/5FU. The toxicological studies including histochemistry and serum biochemistry also demonstrate the biosafety of the orally administered siRNA/5FU formulation and hold a strong promise for pre-clinical studies with larger animals.

### EXAMPLES

Specific exemplary embodiments will now be further described by the following, nonlimiting examples which will serve to illustrate in some detail various features. The following examples are included to facilitate an understanding of ways in which embodiments of the present disclosure may be practiced. However, it should be appreciated that many changes can be made in the exemplary embodiments which are disclosed while still obtaining like or similar result without departing from the scope of embodiments of the present disclosure. Accordingly, the examples should not be construed as limiting the scope of the present disclosure.

### Materials and Methodology

### Materials

The human colon cancer cell line (CaCo2) and stomach cancer cell line (AGS) were purchased from ATCC (Manassas, VA). 5FU, sodium dodecyl sulfate, and Coumarin 6, were purchased from Sigma-Aldrich (St. Luis, MO). β-Glucan (MW 179,000) was purchased from Megazyme (Wicklow, Ireland). Bcl2 siRNA was purchased from cell signaling technology (Danvers, MA). Fetal bovine serum (FBS), penicillin, Phosphate-buffered saline (PBS), and 0.05% Trypsin-EDTA were purchased from Life Technologies (Carlsbad, CA). Bcl-2 SiRNA I (Cat #6441) was purchased from Cell Signaling Technologies (Danvers, MA). Bcl2 antibody, ki67 antibody, and FITC-Goat anti-Rabbit IgG was purchased form ABclonal (Woburn, MA). Simulated gastric juice was purchased from RICCA (Arlington, TX). Click-iT Plus Tunnel assay, eosin, xylene, Harris Hematoxylin, was purchased from Fisher Scientific (Waltham, MA).

### Preparation of BG and Formulations

BG NP were prepared by O/W emulsion method. 30 mg of BG was dissolved in 1 mL of DMSO upon heating at 60° C. and constant stirring for 4 hr. To make BG/5FU, 12 mg of 5FU was dissolved in 1 mL of DMSO and added with the BG solution. Then this DMSO solution was added in 2 mL of DI water slowly drop by drop under high speed of stirring. The DMSO removed by dialysis against water and lyophilized for 24 hr by freeze dryer.

siRNA loaded BG nanoparticle was prepared by W/O emulsion. 30 mg of BG was dissolved in 1 mL of DMSO upon heating at 60° C. and constant stirring for 4 hr. 12 mL of siRNA was dissolved in 1 mL DI water separately. The siRNA solution was added to BG solution drop by drop under vigorous stirring. The formation of the BG/siRNA nanoparticle occurs via electrostatic interaction between BG and siRNA and siRNA locates within the core of the W/O emulsion. The DMSO was removed by dialysis against water and lyophilized for 24 hr upon freeze dryer.

BG/5FU/siRNA nanoparticle were prepared by O/W emulsion. 30 mg of BG was dissolved in 1 mL of DMSO upon heating at 60° C. and constant stirring for 4 hr. 12 mg of 5FU was dissolved in 1 mL of DMSO and added with the BG solution. 12 mL of siRNA was dissolved in 2 mL DI water separately. The siRNA containing DI water was added into the BG and 5FU containing DMSO, drop by drop upon gentle stirring. The DMSO was removed by dialysis against water and lyophilized for 24 hr by freeze dryer. All the formulations were kept at −20° C. while not in use.

Ranges of siRNA concentration would go from 10, 20, 40, 60, 80, 100 nm. Dosage ranged from 1, 3, 5, 10, 20 mg/kg of body weight of mice.

### Characterization of the Formulations

The particle size and zeta potential of BG formulations were determined by a Zeta sizer (Nano ZS, Malvern Instruments, Worcestershire, UK) and kept at 25° C. during the measuring process. All formulations were analyzed three times and the results are presented as the mean. The stability of the formulations and their individual payload were evaluated in saline (pH 7.4) and simulated gastric juice (pH 1-1.4) at different time points. For measuring the size and zeta potential of BG, BG/5FU, BG/siRNA, and BG/5FU/siRNA, all the formulations were diluted in deionized water.

### Stability of the Formulation

To examine the stability of the formulations, naked siRNA and siRNA with BG were diluted in PBS and incubated with BG at 37° C. for 0, 1, 2, 4, and 6 hr, and size and zeta potential were measured. To measure the stability of siRNA in gastric juice with and without BG, siRNA and BG/siRNA were diluted with simulated gastric juice and incubated for 0, 1, 2, 4, and 6 hr and then size and zeta potential were measured according to the method demonstrated earlier. Gel electrophoresis of these formulations was also conducted to confirm the stability of loaded siRNA. To determine the stability of siRNA within BG, the siRNA samples were separated into 1% Agarose gels. Agarose solution was prepared by dissolving in 1×TAE and heated in a microwave for 3 min until dissolved completely. Ethidium bromide was added to the solution before pouring the agarose solution into the casting stand and a 10-well comb was placed to generate wells that are 1.5 mm deep. The melted agarose was allowed to cool for 30 min at room temperature for polymerization. The chamber was filled with 1×TAE buffer solution to a height of 1.5 cm above the gel surface. RNA samples were premixed with the agarose gel loading dye (6×) prior to loading 20 mL (1 μL siRNA, 3 μL dye with 6 μL of simulated gastric juice, and 10 μl BG) of samples into the wells from left to the right. The power supply was activated as soon as all wells were filled, to avoid initial diffusion of the dye into the gel. The samples were run at 100V for 40 min and were imaged using ChmiDoc image analysis (BioRad, USA).

### Development of Stomach Cancer Mice Model

A stomach cancer mice model was developed by feeding N-methyl-N-nitrosourea (NMNU) with water (IACUC protocol no A-201019-A). The 6-week-old C57BL/6 mice were kept in a 12 hr light-12 hr dark cycle. The mice had unprotected access to food and water. NMNU was dissolved in distilled water available ad libitum, freshly prepared three times per week. The mice received 240 ppm NMNU in the drinking water over 5 weeks (every other week). Five weeks after NMNU administration, the mice were ready for the experiment, as the method was reported elsewhere. A total of 30 stomach cancer mice were prepared and divided randomly into 5 groups—no treatment (control), 5FU, BG/5FU, siRNA, BG/siRNA, and BG/5FU/siRNA. Another group of healthy mice were used as a negative control without any treatment. To investigate therapeutic feasibility, 5FU (10 mg/kg) and siRNA (100 nM) was administered either orally (PO) or intravenously (IV). All the formulations were freshly prepared prior to administration. Formulations were administered orally on every 3^{rd} day for the subsequent 5 cycles over 15 days. The animals were sacrificed 72 hr after the last treatment followed by the organ harvest and blood collection for further studies. Immediately after harvesting the size and weight of the stomach and intestine were measured.

### In Vivo Protein Expression

The fresh frozen stomach tissue was homogenized using RIPA buffer with freshly prepared protease inhibitor in it. The homogenate was then centrifuged at 13,000 rpm for 10 min at 4° C. The supernatant was collected, and the total protein content was determined using Bradford's assay (Sigma-Aldrich). SDS-PAGE (12%) was performed to separate protein samples (30 μg) at 70-80 V for 3 hr at room temperature. Later, the proteins were transferred to a PVDF membrane using a transfer unit run at 25 V overnight at 4° C. The PVDF membrane was blocked by incubating with 3% BSA containing TBST for 90 min. The membrane was incubated with Bcl2 and 3-actin primary antibodies (Abclonal, USA) overnight at 4° C. After three washing cycles with ice-cold PBS, the membrane was incubated with horseradish peroxidase-conjugated secondary antibody (Abclonal., USA) for 2 hr at room temperature. The primary and secondary antibodies were used at a 1:1000 dilution ratio. The membrane was treated with ECL mixture. The bands were visualized using a ChemiDoc imaging system (BioRad, USA). The band intensities were calculated using ImageJ software.

### Immunofluorescence and Immunohistochemistry Analysis

Immunofluorescence analysis was performed to understand the Bcl2 and ki67 protein expression in tumor tissues after treatment with different formulations. The 6 m thin sections were obtained by sectioning paraffin-embedded colon tissues using a microtome. Sections were mounted onto poly-1-lysine-coated super plus charged glass slides and rehydrated in multiple concentrations of alcohol and xylene for 5 min each. Sections were incubated with antigen retrieval sodium citrate buffer (10 mM sodium citrate buffer pH 6, 0.05% Tween 20) at 90-95° C. for 10 min and blocked using 2.5% bovine serum albumin (BSA) in Tris-buffered saline for 1 hr at room temperature. Then, the sections were incubated with Bcl2 and ki67 primary antibody (at 1:100 and 1:50 dilution ratio) overnight at 4° C. The next day, the sections were incubated with FITC-Goat Anti-Rabbit IgG secondary antibody for 2 hr at room temperature. The slides were then dehydrated and mounted with DAPI mounting media. Images were acquired using Leica microscope.

### Results and Discussion

### Preparation and Characterization of BG Particles

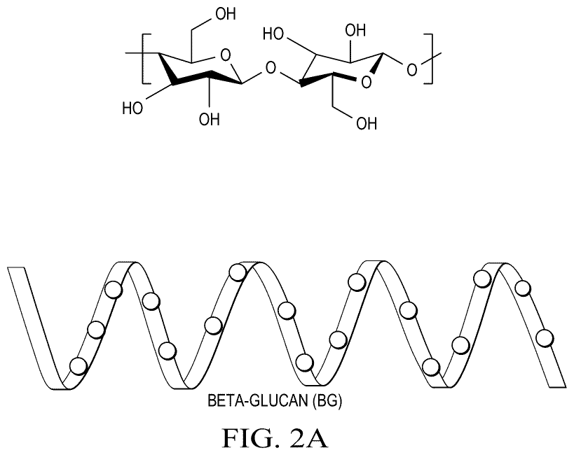

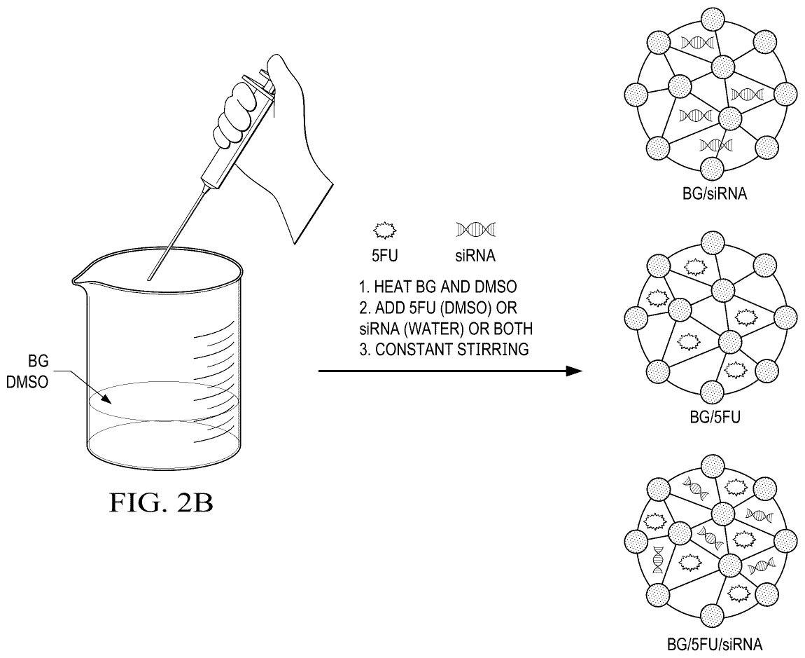

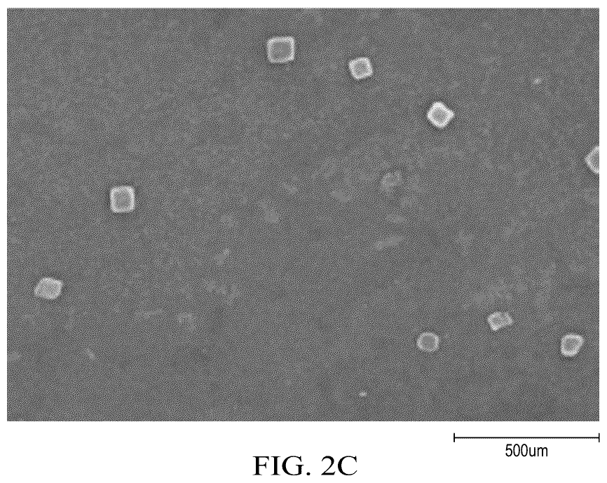

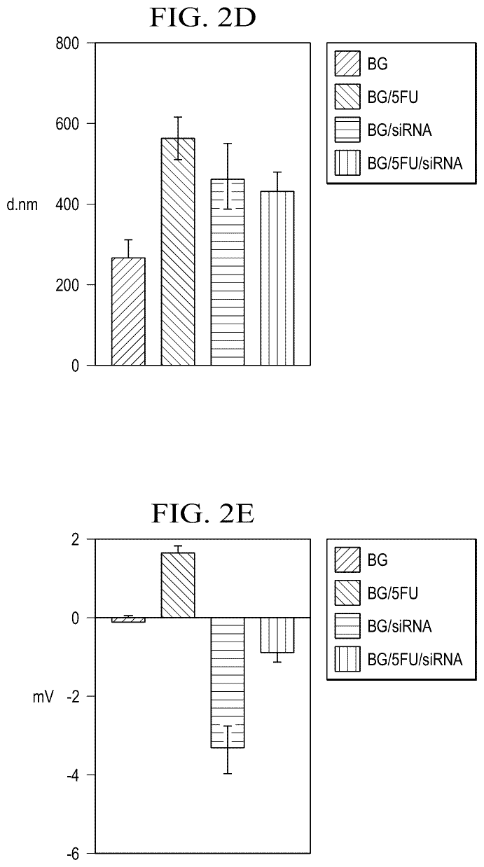

The barley-derived low-viscosity BG used in this study is a long-chain polysaccharide (carbohydrate polymer), with very low solubility in the aqueous solution (FIG. 2A). Though, with gentle stirring in association with rising temperature (up to 70° C.) the BG get dissolved completely in DI water, due to presence of hydrophobic moieties in the backbone, BG forms self-assembled particle while added water at room temperature. We used O/W emulsion method to load hydrophobic 5FU into the core of the BG, and W/O emulsion method to load hydrophilic siRNA. SEM images confirm the shape and size of the—BG/5FU BG/siRNA and BG/5FU/siRNA formulations (FIG. 2C).

The size of BG, BG/5FU, BG/siRNA, and BG/5FU/siRNA was measured as 273.66±39.87, 569±52.43, 470±80.24, 439.66±42.33 respectively. The result indicates that size of the 5FU and siRNA loaded particles are bigger compared to naked BG particles. Zeta potential of BG, BG/5FU/, BG/siRNA and BG/5FU/siRNA were recorded as 0.01±0.06, 1.76±0.09, −3.36±0.63, −0.91±0.19, respectively. The zeta potential of BG/siRNA/siRNA increased compared to BG/siRNA as BG/5FU showed a positive charge (1.76±0.09 mV). The change of zeta potential confirms the successful loading of 5FU and siRNA within BG nanoparticle. We do not see excessive changes, as the siRNA is meant to be loaded within the core and that is not expected to impact on charges on surface.

The size and zeta potential of BG in PBS and simulated gastric juice were measured up to 5 hr of incubation at room temperature. We have observed that the average particle size and zeta potential changed with each loading of the compound. The average size of BG in PBS was 273±39 nm in diameter at the first hour and 386±54 and 583±81 nm in diameter at 3^{rd} and 5^{th} hr of incubation. Which is an indication that the nanoparticle tends to form aggregation and that results in increment of the diameter. Similarly in the presence of simulated gastric juice the average size of BG was found to be 276±15; 399±18; and 608±62 nm in diameter at 1^{st}, 3^{rd} and 5^{th} hr, respectively. It has been observed that with time the size tends to increase, as well. After 5 hr of incubation the size increased by almost 200% in both saline and simulated gastric juice. As the size is almost similar in both PBS and simulated gastric juice, we can conclude that gastric juice does not impact the formulation differently than saline. BG is hydrophilic in nature and due to the presence of abundant hydroxyl groups that participate in hydrogen bonding with water and give the molecule the ability to hold water.

FIGS. 2A-2E show chemical and physical properties of the formulations. FIG. 2A shows chemical structure (repeating unit) and schematic presentation of BG. FIG. 2B shows schematic representation of preparation of formulations; BG/siRNA, BG/5FU and BG/5FU/siRNA. FIG. 2C shows SEM images of BG/5FU/siRNA BG showed smooth surface and nano size (Scale bar=5 μm). FIG. 2D shows size distribution. FIG. 2E shows zeta potential of all the formulations were measured using DLS and zeta analyzer, respectively.

### Mucoadhesive Properties of BG

FIG. 2A demonstrates how BG particles interact with mucus layer within stomach. The mucoadhesiveness and intestinal transportation of hydrophobic small molecule were further confirmed by an ex vivo diffusion study in a porcine intestine lumen. As the mucous layer is a barrier for hydrophobic molecules, to check how it crosses through the epithelial layer with assistance of BG we have used coumarin-6, a small molecule hydrophobic fluorescent dye with similar chemical and physical properties (molecular weight and hydrophobicity) as 5FU, for ease of visualization and detection purpose. Higher fluorescence intensity and accumulation of coumarin-6 was observed in the stomach layer with increment of duration of incubation. At the 2^{nd} hr, presence of coumarin-6 was observed within deeper layers of the intestine, which indicates the transportation of hydrophobic small molecule (FIG. 2B). At the 12^{th} hr, coumarin-6 incorporates evenly across the layers of the intestine. The same tissue was subjected to be observed under a ChemiDoc imager to capture the fluorescence signal within the cross-sectioned of the intestinal lumen and similar result was observed. With time (30 min, 2 hr, and 12 hr) more coumarin-6 were distributed throughout the intestine which is an indication and confirmation of BG's ability to facilitate intestinal transportation of hydrophobic payload (FIG. 2C). However, the image with only cumarin-6 shows that without BG cumarin-6 itself is not able to diffuse through the intestinal layer.

Mucoadhesive polymers have been found to be crucial in delivering nucleic acid and peptide drugs. Recently a study stated that mucoadhesive nanoparticle has the potential to deliver KDM6A-mRNA on the targeted site and treat bladder cancer. SPR analysis was performed to examine how BG interacts with mucin. The results demonstrate that BG has mucoadhesive properties and binds with the mucus in presence of acidic simulated gastric juice. A carboxylated gold (Au) chip was modified with BG and a solution of mucin was run over the chip to understand their binding kinetics, association, and dissociation profile. SPR analysis shows a response of 30000 RU from baseline, which confirms the strong binding affinity of BG with mucin. Curve (a) indicates the association of BG with mucin, curve (b) represents the plateau as it continues binding, and curve (c) represents the dissociation of BG with mucin. As mucin is the main component of the mucus layer, the association, and binding profile of BG with mucin provides evidence of BG's affinity with mucus layer and its ability to bind with (FIGS. S3 and S4).

Stability of siRNA Payload in BG Formulation

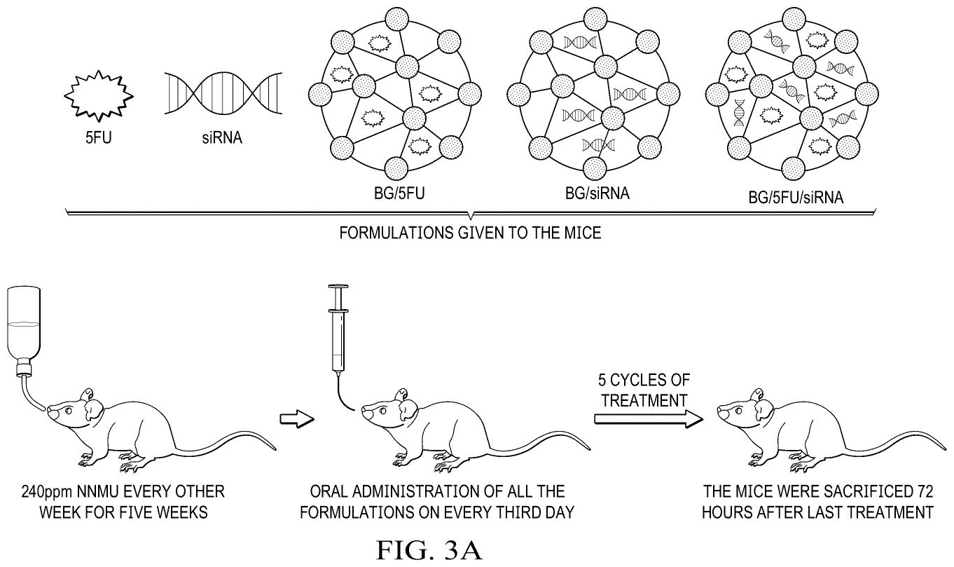

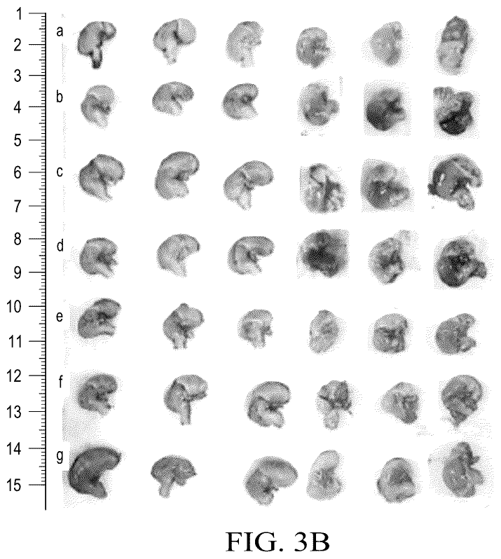

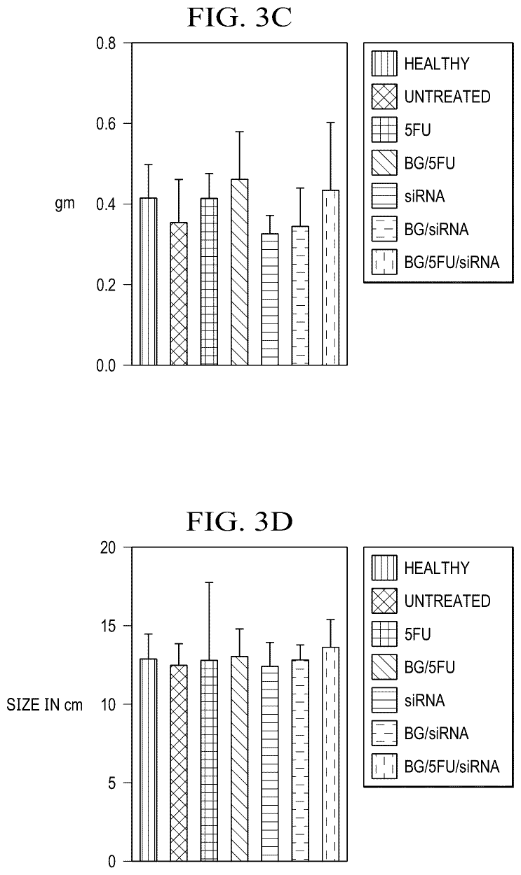

The stability of siRNA was measured with and without BG in PBS and simulated gastric juice. The size and zeta potential measurement of BG and BG/siRNA in presence of PBS and simulated gastric juice were conducted from 0 to 6 hours to examine the stability of siRNA payload. siRNA is a nucleic acid with negatively charged surface and forms particles via electrostatic interaction with the partially cationic-charged BG (0.0158 mV). Size of the BG/siRNA formulation was measured as 370±80 nm at 0 hr and 707±76 nm in diameter at 6^{th} hr. The increasing size indicates the possibility of self-aggregation of siRNA in simulated gastric juice when attached to BG (FIGS. 3B and 3C). The result was confirmed by zeta potential measurements. As negatively charged siRNA was attached to positively charged BG the net charge of the BG/siRNA became almost neutral. The zeta potential of BG/siRNA in simulated gastric juice was measured as −0.68±0.99 mV immediately after dissolve and −0.80±1.96 mV after 6 hours of incubation (FIGS. 3E and 3F). The consistency of the zeta potential confirms the stability of siRNA payload within BG while dissolved into simulated gastric juice.

The stability of siRNA was further confirmed by the agarose gel electrophoresis. The band shows that the siRNA is stable while loaded within BG for at least 6 hours both in PBS and simulated gastric juice (FIG. 3D).

### Phenotypical In Vivo Treatment Efficiency

Stomach cancer was induced to C57BL/6 mice by feeding N-methyl-N-nitrosourea (NMNU) in every other week for 5 weeks period. The cancer induced mice were treated with saline, 5FU, siRNA, BG/5FU, BG/siRNA, and BG/5FU/siRNA via oral administration for 5 times at every 3^{rd} day. To investigate phenotype of therapeutic progress upon treatment with formulation in compared with saline treated mice, we have harvested the animal after 72 hr of 5^{th} treatment. The size and weight of the stomach and intestine were measured immediately after the collection of the organs. A reduction in the size and weight of the stomach was observed in the untreated and oral siRNA treatment group, and this is an indication of a cancerous state. However, no significant difference in the size and weight of the intestine was observed, which is an indication of localized cancer specific to the stomach. In stomach cancer, the mice loss appetite due to reduced production of ghrelin hormone, hence we have observed weight loss in the untreated group when compared to the healthy control. On top of that, cancer-mediated inflammation also results in the shrinkage of the organ, the stomach in this case. Reduction of size upon oral administration of nitroso compound is one of the phenotypic indications of induction of stomach cancer, and gaining the size back to normal which is comparable to healthy mice demonstrated the therapeutic efficacy of orally administered BG/siRNA/5FU formulation. The therapeutic efficacy of BG/siRNA/5FU has been further confirmed with histological and immunohistichemistry analysis with the tissues.

Oral Bcl2 siRNA Causes In-Vivo Gene Silencing and Apoptosis

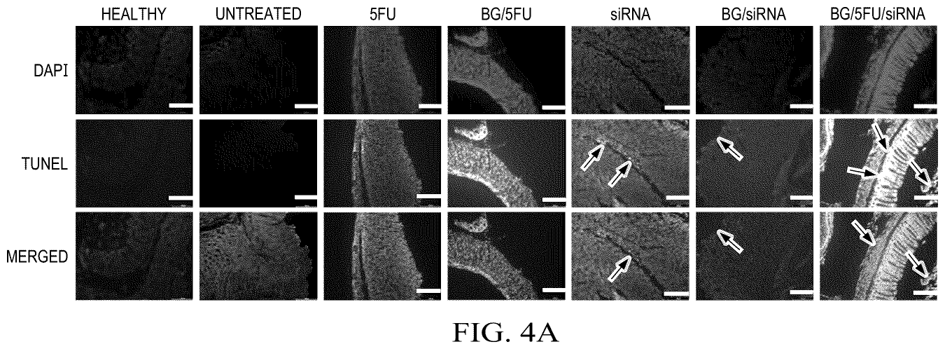

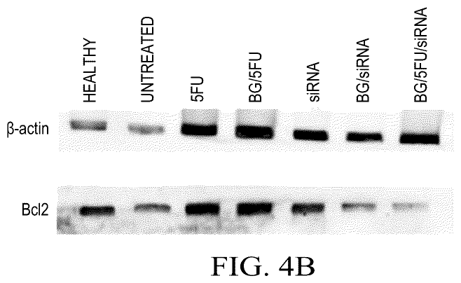

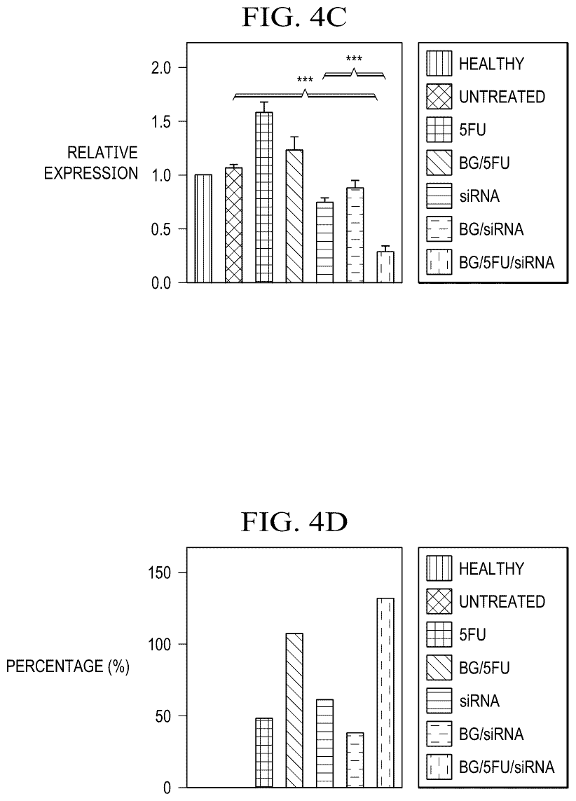

A TUNEL assay was conducted to investigate the apoptosis within the tissue from the stomach of both healthy, untreated cancer, and treated cancer mice. We have observed significantly higher apoptosis in the stomach treated with BG/5FU/siRNA than the stomach treated with naked siRNA and both untreated and healthy control (FIGS. 4A and 4D). No fragmented DNA was observed within the stomach tissues of healthy control and untreated mice, which indicates that oral co-delivery of siRNA and 5FU induces apoptosis in stomach cancer. Apoptosis was quantified by the mean fluorescent intensity and compared with healthy control and untreated control. The degree of apoptosis was calculated as 49, 107, 38, 62, and 131% for the 5FU, BG/5FU, siRNA, BG/siRNA, and BG/5FU/siRNA group relative to untreated control, respectively.

FIGS. 3A-3D show in vivo therapeutic efficacy. FIG. 3A shows a schematic of animal model preparation and treatment. FIG. 3B shows an image of the stomach collected from different group of treatment—Healthy (row a), Untreated cancer (row b), BG/5FU (row c), 5FU (row d), siRNA (row e), BG/siRNA (row f), BG/5FU/siRNA (row g). FIG. 3C shows weight of the stomach. FIG. 3D shows size of the stomach.

This reveals that apoptosis for BG/5FU/siRNA treated group was 69.46% more than BG/siRNA, and 95% more than free siRNA. To examine if the apoptosis was caused by downregulation of Bcl2, western blotting (WB) analysis was performed from the tissue lysate of different treatment groups. The WB data shows significant downregulation of Bcl2 gene resulted in by oral BG/5FU/siRNA compared to conventional naked oral siRNA (p-value<0.005), and untreated (p-value<0.0005). Data showed that the downregulation of Bcl2 gene expression was inversely proportional with the DNA fragmentation, which confirmed that the apoptosis resulted from the Bcl2 downregulation. Relative expression of Bcl2 was 1; 1.07±0.008; 1.24±0.11; 0.76±0.02; 0.88±0.05; 1.66±0.14; 0.29±0.04 for the healthy, untreated, 5FU, BG/5FU, siRNA, BG/siRNA, and BG/5FU/siRNA, respectively. This data confirms that BG-assisted orally administered siRNA was protected from the harsh gastric environment and was able to pass the mucus layer of the intestine and go into the site of action to silence the Bcl2 gene.

FIGS. 4A-4D show immunohistochemistry to attribute apoptosis. In FIG. 4A, tunnel assay shows broken nucleus (arrow) in siRNA, BG/siRNA, and BG/5FU/siRNA groups. Scale bars represent 200 μm. In FIG. 4B, western blot analysis also shows reduced Bcl-2 expression in BG/5FU/siRNA group compared to naked siRNA and BG/siRNA. The error bar represents the standard deviation. FIG. 4C shows quantitative analysis of western blot. FIG. 4D shows apoptosis in percentage. P value<0.0001 is denoted as ***.

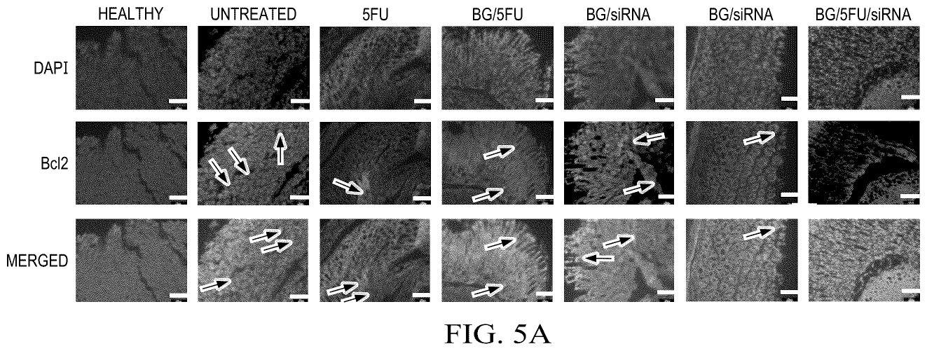

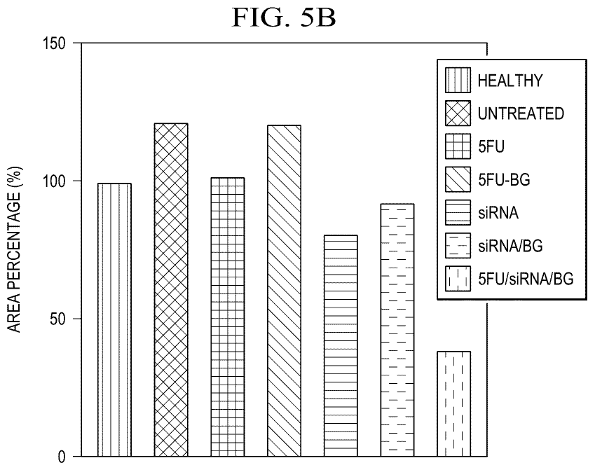

The stomach tissue was also utilized to analysis Bcl2 protein expression using immunohistochemistry (IHC). The anti-Bcl2 positive area was quantified in comparison to the whole tissue section stained with DAPI. The area percentage of Bcl2 antibodies for healthy, untreated, 5FU, BG/5FU, siRNA, BG/siRNA, and BG/5FU/siRNA was 1.19%, 1.46%, 1.22%, 1.45%, 0.97%, 1.11%, 0.47% (FIG. 5A). To measure the relative expression of Bcl2 on the tissue section the data was calculated and compared with the stomach tissue collected from the healthy mice, where Bcl2 expression in healthy mice was considered as 100% (FIG. 5B). The immunofluorescence data reciprocate the same as WB data. Oral delivery of BG/5FU/siRNA resulted in the lowest expression of Bcl2 as observed in WB and immunofluorescence images of tumor tissue. In the immunofluorescence image, we have observed Bcl2 expression (20%) in the tissue treated with BG/5FU/siRNA in compared with tissue from untreated tumor bearing mice. The mice treated with BG/5FU/siRNA had 83% less expression of Bcl2 compared to untreated cancer and 42% less expression than mice treated with naked siRNA. Mice treated with BG/siRNA also causes less expression of Bcl2 confirmed by WB and ICH but the combination of siRNA and 5FU resulted in higher Bcl2 inhibition efficiency. This confirms that siRNA with 5FU produces a synergistic effect in downregulating the Bcl2 proteins.

FIGS. 5A-5B show immunohistochemistry to attribute expression of Bcl2 protein. In FIG. 5A, immunohistochemistry for Bcl-2 protein shows higher expression of Bcl-2 in untreated mice stomachs (red arrow). The naked oral siRNA treatment group also had more Bcl-2 expression than BG/siRNA and BG/5FU/siRNA. The scale bar represents 100 μm. In FIG. 5B, quantification of Bcl2 immunofluorescence data analysis was presented.

### Therapeutic Efficacy In Vivo

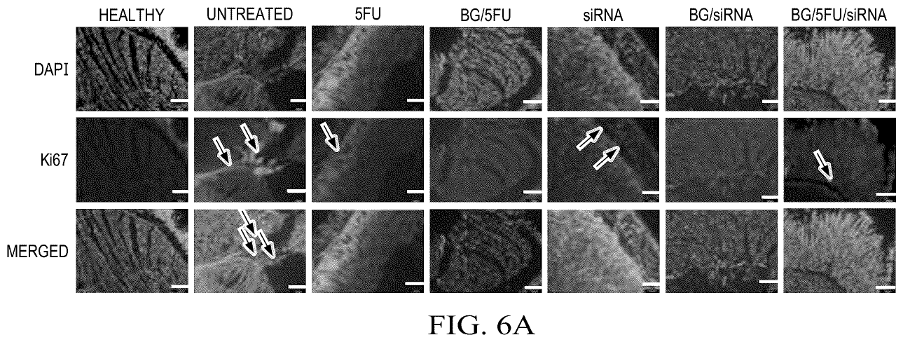

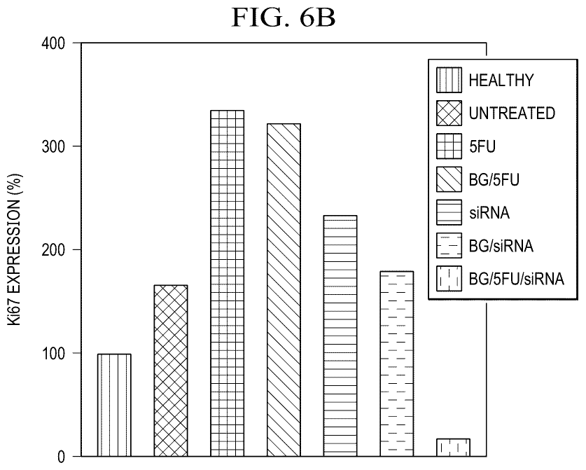

Cell proliferation marker was measured by immunofluorescence assay to evaluate the antitumor effect of the formulations. Ki67 is a protein found in the nucleus of the cancer cell. Detection of Ki67 expression has been a reliable indicator for cancer proliferation and prognosis in cancers including stomach cancer. Therefore, Ki67 is considered an important indicator of therapeutic assessment and chemosensitivity. Studies show that higher expression of Ki67 corresponds with a low chemosensitivity. In this study, the stomach section from different treatment groups of mice was subjected to immunofluorescence for Ki67 protein analysis. The untreated stomach showed higher expression of Ki67 (yellow arrow) and with treatment, specifically with BG/5FU/siRNA, the expression of Ki67 expression was reduced significantly (FIG. 6A). The protein expression was quantified from immunofluorescence images. The area with Ki67 positive cells was measured in compared to the whole tissue section with DAPI stained. The area (%) of healthy, untreated, 5FU, BG/5FU, siRNA, BG/siRNA, and BG/5FU/siRNA was measured as 0.16%, 0.27%, 0.54%, 0.52%, 0.38%, 0.29%, and 0.03%, respectively. The Ki67 expression in the untreated group was 68.75% higher in the untreated group compared to the healthy control. Whereas, with BG/5FU/siRNA treatment group the expression was reduced by 150% when compared to the stomach of untreated cancer mice (FIG. 6B). We have also observed partial expression of Ki67 to the group of mice treated with 5FU which is comparatively higher than mice treated with BG/5FU/siRNA.

FIGS. 6A-6B show immunofluorescence for ki67 of stomach sections of different treatment groups. FIG. 6A shows that a higher expression of ki67 (arrow) was observed in untreated group, whereas after treatment the expression got reduced specially with BG/5FU/siRNA formulation. Scale bar represents 100 μm. FIG. 6B shows that Ki67 expression was quantified compared to healthy control.

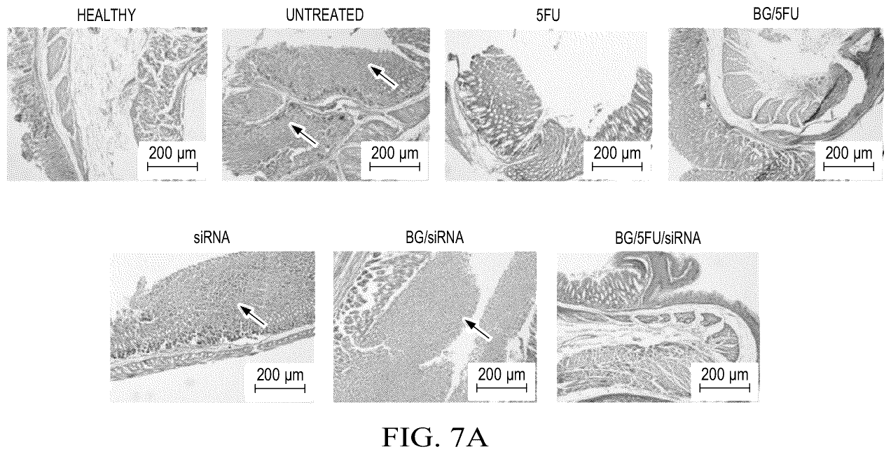

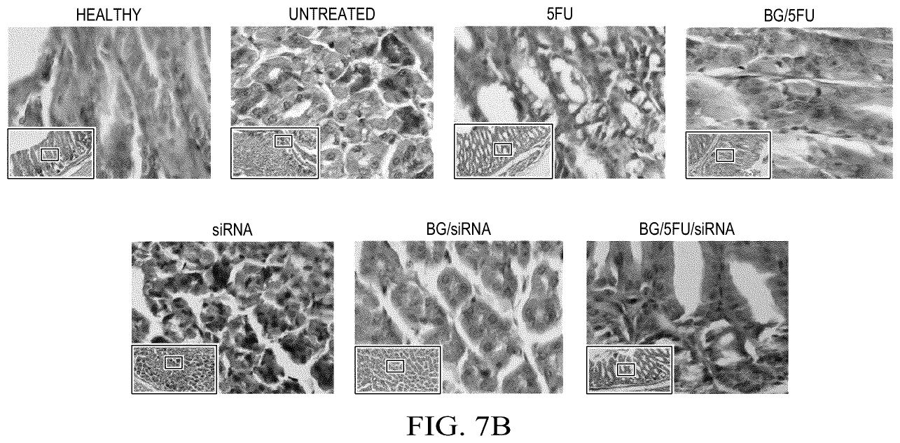

H&E staining with stomach tissue was conducted to measure the anticancer therapeutic effect of stomach cancer as well as the inflammatory effect among the tissue/cell around the cancer. FIGS. 7A-7B represent the H&E staining image of the same stomach section in different magnifications. To visualize the whole tumor area the image was taken at 10× magnification and to understand the pathophysiology a higher magnification image was taken at 40× by a slide scanner. As seen in FIGS. 7A-7B, the presence of tumors in the stomach as indicated by the black arrow. Moreover, a large intramucosal tumor was observed in the stomach of untreated cancer mice, and cancer mice treated with siRNA and BG/siRNA. However, no visible tumor was observed in the stomach of the cancer mice treated with BG/5FU/siRNA. The total remission of cancer indicates the ability and anticancer efficacy of oral delivery of the 5FU/siRNA assisted by BG.

FIGS. 7A-7B show histology of stomach. In FIG. 7A, the histological staining (H&E) shows a growth (arrow) in the untreated section, while after treatment with 5FU, 5FU/BG and BG/5FU/siRNA the tumor region got significantly reduced. FIG. 7B shows H&E staining of stomach cancer sections with 40× magnification. The rectangular area was cropped and magnified for better morphological understanding. The untreated, naked siRNA and BG/siRNA shows the feature of adenocarcinoma. The section of BG/5FU/siRNA treatment group almost resemblance as healthy stomach.

In FIG. 7B, the images were taken at 40×, and then the rectangular area was cropped to magnify for better and clearer visualization of the tissue morphology. The higher magnification of the images shows adenocarcinoma in the untreated group where there was higher staining of nuclei, irregular glandular space, and distortion of normal epithelial structure. Signet ring cell was also found in the untreated stomach cancer tissue. No improvement of tumor was observed with naked siRNA treated stomach section. However, mice treated with BG/5FU/siRNA and BG/5FU demonstrate that the cellular structure almost resembled to healthy stomach.

### Serum Biochemistry and Toxicity

To examine the systemic side effect mediated by the treatment modalities, serum analysis was conducted with the blood collected from both healthy controls, untreated, and treatment groups. Liver and kidney toxicity was measured by quantifying the presence of total protein (TP), alanine aminotransferase (ALT), creatinine, and blood urea nitrogen (BUN) in blood and compared with healthy control. None of the parameters were found out of the reference range, which confirms that the oral formulations are not significantly toxic to the liver and kidney. As the treatments were given orally, the H&E staining of the small intestine was also done, and no toxicity was observed in the histological section of the duodenum, jejunum, and ilium of the different treatment groups.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition of matter for treatment of stomach cancer, comprising:
fluoreuracil-5; and
Bcl2 siRNA.

2. The composition of claim 1, further comprising at least one mucoadhesive nanocarrier.

3. The composition of claim 2, wherein the at least one mucoadhesive nanocarrier comprises at least one carbohydrate polymer.

4. The composition of claim 3, wherein the at least one carbohydrate polymer comprises at least one biocompatible polysaccharide.

5. The composition of claim 4, wherein the at least one biocompatible polysaccharide comprises at least one β-glucan.

6. A method of treatment of stomach cancer, comprising:
administering to a mammal in need thereof a compound including
fluoreuracil-5; and
Bcl2 siRNA.

7. The method of claim 6, wherein administering comprises oral administration.

8. The method of claim 7, wherein administering includes administering the compound wherein the compound includes at least one mucoadhesive nanocarrier.

9. The method of claim 8, wherein administering includes administering the compound wherein the at least one mucoadhesive nanocarrier comprises at least one carbohydrate polymer.

10. The method of claim 9, wherein administering includes administering the compound wherein the at least one carbohydrate polymer comprises at least one biocompatible polysaccharide.

11. The method of claim 10, wherein administering includes administering the compound wherein the at least one biocompatible polysaccharide comprises at least one β-glucan.

12. A method of oral treatment of disease, comprising:
administering to a mammal in need thereof a compound comprising
fluoreuracil-5; and
Bcl2 siRNA.

13. The method of claim 12, wherein administering includes administering the compound wherein the compound includes at least one mucoadhesive nanocarrier.

14. The method of claim 13, wherein administering includes administering the compound wherein the at least one mucoadhesive nanocarrier comprises at least one carbohydrate polymer.

15. The method of claim 14, wherein administering includes administering the compound wherein the at least one carbohydrate polymer comprises at least one biocompatible polysaccharide.

16. The method of claim 15, wherein administering includes administering the compound wherein the at least one biocompatible polysaccharide comprises at least one β-glucan.
